(19) United States
(12) Patent Application Publication
(10) Pub. No.: US 2012/0100040 A1
(43) Pub. Date: Apr. 26, 2012

(54) COMPOSITION FOR STERILIZING SURFACES

(75) Inventors: Bjørg Marit ANDERSEN, Oslo (NO); Erik Edvin Berg, Oslo (NO)

(73) Assignee: Bakteriefritt AS, Oslo (NO)

(21) Appl. No.: 13/284,376

(22) Filed: Oct. 28, 2011

Related U.S. Application Data
(63) Continuation-in-part of application No. PCT/NO2010/000150, filed on Apr. 23, 2010.

(30) Foreign Application Priority Data
Apr. 30, 2009 (NO) 20091724

(51) Int. Cl. A61L 2/18; A01P 1/00; A01N 59/00

(52) U.S. Cl. 422/28; 424/616

(57) ABSTRACT

Compositions and methods for disinfesting rooms and objects through a mist are disclosed. The compositions generally include a bactericidic and/or parasiticidic substance, a poly-acid or hydrophilic sulfone, and optionally a cell membrane polarizing component. By using an organic poly-acid or hydrophilic sulfone in a composition containing the bactericidic and/or parasiticidic substance, such as hydrogen peroxide or akacid, for disinfecting rooms and objects through a mist of said composition, it is possible also to kill mycobacteria.

### CROSS-REFERENCE TO RELATED APPLICATIONS

This application claims priority to International Application No. PCT/NO2010/000150, titled COMPOSITION FOR STERILIZING SURFACES, filed on Apr. 23, 2010, which claims priority to Norwegian Application No. 20091724, filed on Apr. 30, 2009, the disclosures of which are hereby incorporated by reference herein in their entireties.

### BACKGROUND

Within the health and care sector (hospitals, nursing homes, homes for the elderly, care homes for the mentally disabled, acute receptions, operating rooms, etc.) contagious diseases spreading through droplets, touch, transfer through objects that are delivered by hand from persons with the relevant disease and healthy persons alternately, air-borne spores, virus, etc. may lead to serious contagious disease conditions. Microorganisms (bacteria, virus, mycoplasma, fungi, spores, etc.) and parasites (mites, lice, etc.) may represent a problem both concerning removal and concerning representing a continuous danger for contagious diseases for persons living and working under such environments, or that come in contact with the relevant surfaces or objects or treat surface wounds having been exposed to such microorganisms. Particularly in hospitals the danger of catching a contagious disease is large, especially concerning multi-resistant or empowered medication-resistant bacteria by the growth of such bacteria being favored by other “normal” bacteria becoming reduced through antibiotics.

Registered data show that more than 60,000 persons get infections of different kinds of diseases in Norwegian hospitals per year, and about 4,000 die from this. Hospital acquired infections (HAI) account for thousands of extra hospital days and these infections are also spread between hospitals, care centers and elsewhere in the society. Statistically, the infection frequency in Norwegian hospitals is 5.5% and the infection frequency in the Norwegian care center sector is about 7-8%. In addition, there also exists large dark numbers. Disinfection of rooms in hospitals, care centers, nursing homes and similar institutions is mainly performed as a preventive measure for reducing the transmission of diseases in such locations.

An example of such conditions is the arising of multi-drug resistant (MDR-TB) and extra drug-resistant (XDR-TB) tuberculosis bacteria in hospitals, wherein the tuberculosis bacteria are resistant to known antibacterial drugs (isoniazid, ethanmutol, pyrazinamide and rifampin as first-row drugs and combinations thereof as second-row drugs) [Scientific American, March 2009, p. 56-63]. However, other infections may be airborne and difficult to handle as well, e.g. infections with bacteria from the genus Pseudomonas, Staphylococcus, Aspergillus, etc., nosocomial infections, yeast infections, etc.

Within areas with a large need for non-bacterial conditions (operating theatres, sterile areas, reconvalesence areas, etc.) anti-bacterial treatment of visible surfaces alone or treatment with mild antiseptic substances is not sufficient.

Surface injuries (fire victims, allergic persons, electrocuted victims, persons with ablations, etc.) in particular are susceptible to infections by the above mentioned bacteria and microorganisms, and such injuries are notoriously difficult to treat on account of their delicate nature and the need for keeping the remaining skin and damaged areas moist while keeping the danger of infections reduced to a minimum. In such injuries the comfort of the patient is a concern, and supplying hydrogen peroxide-containing fluids directly by brush or spray to areas where the skin has been breached or removed can result in unbearable stinging and burning sensations. On account of the danger for catching drug-resistant bacterial or yeast infections or viral infections, such injuries represent a large problem and concern for the doctors treating them.

In connection with decontaminating surfaces and objects it is also of importance that non-reachable and/or non-visible areas and surfaces are disinfected/decontaminated. This concerns e.g. cracks, niches, pipes, tubes, etc. where it is difficult/impossible to reach the surface with conventional spraying or brushing techniques. Since conventional spraying or brushing techniques both represent methods where only surfaces in the line of sight (or at best in a limited way around corners with a bent brush) are reached, such methods are not suitable for a total disinfection of rooms or objects. Also conventional spraying or brushing techniques leave the treated surface wet or moist, and such fluid has to evaporate before the surface again may be used for its intended purposes. This moisturizing effect also has as a consequence that such methods are unsuited for disinfecting delicate electronic equipment, e.g. in hospitals. It will furthermore be advantageous to treat such surfaces with substances that are non-toxic, that do not smell, that do not leave residues on the treated surfaces and that do not require long shut-down periods of important apparatuses (e.g. machines in operating theatres of life-supporting machines in reconvalescence rooms).

The removal of microorganisms from surfaces and objects has previously been conducted by washing the relevant surfaces with disinfecting substances such as hydrogen peroxide (H₂O₂), akacid+, ammonium chloride (NH₄Cl), formaldehyde, glutaraldehyde, orto-formaldehyde, potassium persulfate, amidosulfonic acid, sodium perborate, alcohols, peracetic acid or combinations thereof, to mention some. However, a brushing of such disinfecting substances alone will not be sufficient to apply such substances to poorly reachable surfaces and areas.

Previously it is known to spray an aqueous mist of an antimicrobial substance to distribute this evenly over the relevant surface and/or object and additionally access the unreachable areas. For example, US patent application 2007/0125882 discloses spraying a “dry” mist of aqueous hydrogen peroxide onto surfaces to disinfect the surfaces. An aqueous hydrogen peroxide composition with a H₂O₂-concentration in the interval 3-5% (v/v) is used. The oxidizing effect of H₂O₂ attacks membranes and DNA-RNA.

An example of efficiency when treating a room with such a dry spray mist of hydrogen peroxide is shown in FIGS. 1 and 2. These figures show the number of deaths from the bacterium Clostridium difficile (“Infection and Control University Hospitals of Leicester NHS Trust”). FIG. 1 shows the number of deaths before disinfection with such a dry spray mist of hydrogen peroxide was introduced. FIG. 2 shows the reduction of deaths after treatment of the environment with hydrogen peroxide mist was introduced.

However, it has been found that such a mist, albeit effective towards most bacteria and microorganisms, is not active towards mycobacteria. This is a serious disadvantage since many contagious and dangerous diseases are caused by mycobacteria. An example having been pointed out supra is tuberculosis (Mycobacterium tuberculosis) (MDR-TB and XDR-TB). Another example is pneumonia-causing mycobacteria (Mycobacterium pneumonia) being a dangerous and contagious disease for infants and especially for elderly people. Other examples of mycobacterium infections in humans being difficult to handle with conventional means, are infections caused by Mycobacterium ascessus, and also animals may be attacked by mycobacteria (e.g. Mycobacterium bovis or Mycobacterium avis). Such infections may exist, e.g. at veterinarians, where sterile conditions also may be of importance, e.g. in operating theatres. Since patients in hospitals already may suffer from a poor general condition and weakened immune defenses (e.g. through the use of cell poison based on other diseases, for example HIV), it is of special importance that disinfection removes all bacteria and microorganisms. As explained supra a partial removal of bacteria or other sources of disease may actually compound the problems concerning infections in hospitals.

One theory for the poor efficiency of hydrogen peroxide spray towards mycobacteria is based on mycobacteria having a cell wall structure existing as a complicated membrane being impenetrable for H₂O₂ in the form of a spray mist. This opens, as explained supra, for selective colonization by mycobacteria on the relevant surfaces, something that may represent an even worse situation than prior to the spray treatment. One of the reasons for the very effective defense in mycobacteria is, as explained supra, the io complex structure of the cell wall surface being formed by a complex structure of peptide glycans, arabino galactane, mycolate, acyl, lipids (LAM, lipo arabino mycholate) outside the lipid bilayer of the cell membrane, giving a surface where water and aqueous compositions are rejected and where the surface structure has distributed penetrating porins. Water-based spray mist (e.g. water-based hydrogen peroxide mist or akacid+, poly-guanidine) will consequently not penetrate this surface structure.

Consequently there exists a strong need for an improved aqueous disinfecting formulation that is effective against mycobacteria and bacterial spores, which similar to mycobacteria, can be very difficult to kill using conventional decontamination and/or sterilization techniques.

### SUMMARY

A composition and process suitable for killing microorganisms and parasites is disclosed, wherein the composition is supplied to surfaces in the environment or surfaces on living organisms such as humans or animals, e.g. the dermis/skin and/or fur/hair of such organisms, in the form of a “dry” spray or mist being sprayed to the environment from a nozzle. The antibacterial and/or antiparasitic aqueous compositions according to the present disclosure include a bactericidic or parasiticidic substance, such as hydrogen peroxide or akacid, and may include or be combined with a cell membrane-opening substance, in particular a mycobacterium cell membrane-opening substance, functioning as an excipient that penetrates the cell wall structure/surface structure of the bacteria, virus, fungi, parasite, and/or spore and assures that an attack from the bactericidic or parasiticidic substance on the cell/cell membrane is effective. The cell membrane opening substance may be a non-toxic hydrophilic organic poly-acid, e.g. citric acid, or a hydrophilic sulfone such as a di-C₁₋₈-sulfone, di-C₁₋₆-sulfone, di-C₁₋₅-sulfone, di-C₁₋₄-sulfone, di-C₁₋₃-sulfone, di-C₁₋₂-sulfone, or dimethylsulfone. The composition can also include a polarizing agent, such as a metal ion, to aid in the depolarization of cell membranes.

Another aspect of the present disclosure relates to the use of a hydrophilic sulfone, such as di-C₁₋₈-sulfone, di-C₁₋₆-sulfone, di-C₁₋₅-sulfone, di-C₁₋₄-sulfone, di-C₁₋₃-sulfone, di-C₁₋₂-sulfone, or dimethylsulfone, and/or a non-toxic hydrophilic organic poly-acid, such as citric acid, as an excipient for opening the cell membrane of mycobacteria for simultaneous or subsequent attack against such mycobacteria from at least one bactericidic and/or parasiticidic material, such as hydrogen peroxide and/or akacid (poly-guanidine). Such a use can include the addition of citric acid and/or dimethylsulfone to a “dry” spray mist of a bactericidic or parasiticidic material, e.g. hydrogen peroxide and/or akacid, separately, prior to, or simultaneous with the aqueous antimicrobial or antiparasiticidic mist. In an embodiment, the mycobacterium cell membrane opening substance may be added to the sprayable aqueous antimicrobial or antiparasiticidic composition prior to the spraying.

Another aspect of the present disclosure is a process for sterilizing surfaces with a composition according to the present disclosure, said process optionally including a neutralizing step for the dry hydrogen peroxide mist subsequent to ending the antimicrobial treatment.

Yet another aspect of the present disclosure includes a device and process for decontaminating or disinfecting the treatment compartment in emergency vehicles such as ambulances, wherein said device comprises an inflatable seal to be placed in association with the door jambs and abutments for making at least the treatment compartment of the vehicle air-tight for subsequent treatment of said compartment with a hydrogen peroxide-containing dry mist, according to the present disclosure.

### BRIEF DESCRIPTION OF THE DRAWINGS

FIG. 1 is a diagram illustrating the number of deaths before disinfection.

FIG. 2 is a diagram illustrating the reduction in the number of deaths after treatment of the environment with a dry mist of hydrogen peroxide.

FIG. 3 is a rear cross-sectional view of an example compartment, such as a sweat box.

FIG. 4 a side cross-sectional view of the example sweat box shown in FIG. 3.

FIG. 5 is a perspective view of an example sealing device.

FIG. 6 is a side view of the example sealing device.

FIG. 7 is a perspective view of an example vehicle in which the sealing device shown in FIGS. 5-6 can be secured.

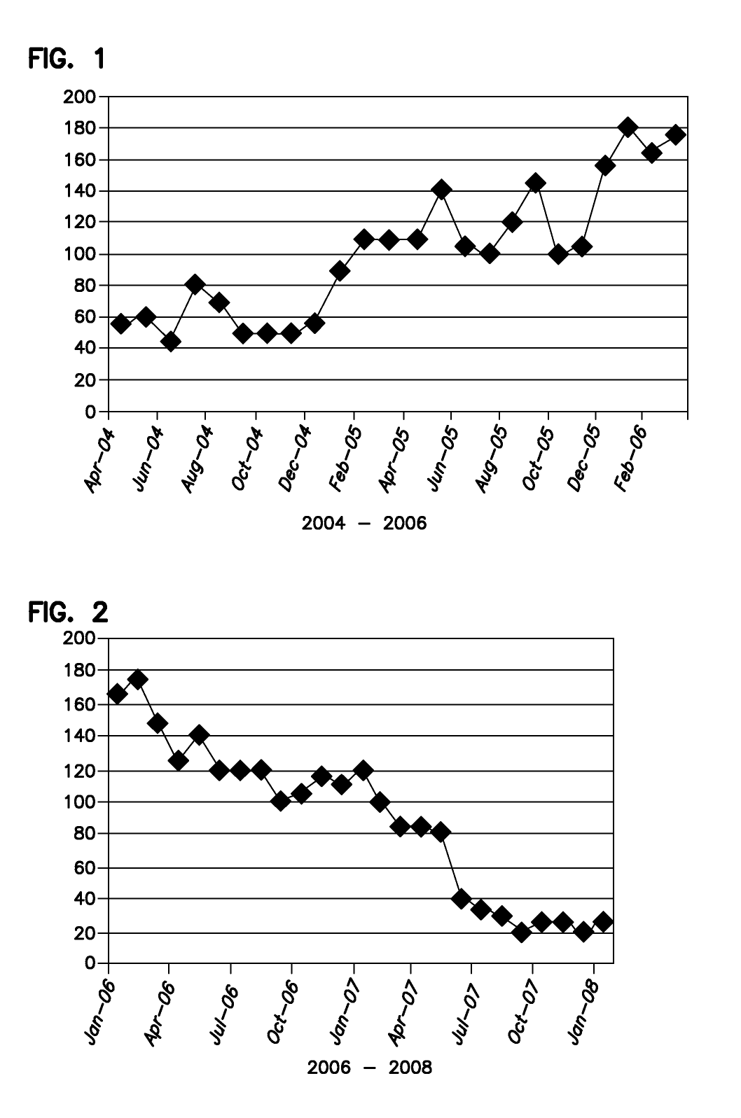

### DETAILED DESCRIPTION

Within the health and care sector (hospitals, nursing homes, homes for the elderly, care homes for the mentally disabled, acute receptions, operating rooms, etc.) contagious diseases spreading through droplets, touch, transfer through objects that are delivered by hand from persons with the relevant disease and healthy persons alternately, air-borne spores, virus, etc. may lead to serious contagious disease conditions. Particularly in hospitals, the danger of bacterial infection is high, especially from multi-resistant or empowered medication-resistant bacteria. Hospital acquired infections (HAI) account for thousands of extra hospital days and these infections can be spread between hospitals, care centers and elsewhere in society.

is The removal of microorganisms from surfaces and objects in the heath and care sector has previously been conducted by washing the relevant surfaces with disinfecting substances, such as hydrogen peroxide (H₂O₂), akacid, akacid plus, ammonium chloride (NH₄Cl), formaldehyde, glutaraldehyde, orto-formaldehyde, potassium persulfate, amidosulfonic acid, sodium perborate, alcohols, and peracetic acid. However, brushing of such disinfecting substances alone is not sufficient to apply such substances to poorly reachable surfaces and areas. The spraying of an aqueous mist of an antimicrobial substance, such as hydrogen peroxide, to distribute the antimicrobial substance evenly over the relevant surface and/or object and treat areas unreachable by brushing is known. See, for example, US patent application 2007/0125882. It has been found, however, that hydrogen peroxide sprays have poor efficiency towards mycobacterium and, in some instances, bacterial spores.

The present disclosure describes a solution to the above mentioned problem with survival of mycobacteria and bacterial spores by adding to the aqueous bactericidic and/or parasiticidic composition a substance that opens up the surface and membrane structure of bacteria, viruses, fungi, parasites, and spores so that a bactericidic and/or parasiticidic substance, such as hydrogen peroxide or akacid, can penetrate into the bacteria, virus, fungus, parasite, or spore and attack components such as membranes, internal bacterial structures and DNA. Examples of such substances include, but are not limited to, a non-toxic hydrophilic organic poly-acid, a hydrophilic sulfone, and mixtures thereof.

The bactericidic and/or parasiticidic substance includes, but is not limited to, hydrogen peroxide, akacid, akacid plus, poly-guanidine, ammonium chloride (NH₄Cl), formaldehyde, glutaraldehyde, orto-formaldehyde, potassium persulfate, amidosulfonic acid, sodium perborate, alcohols, peracetic acid, and mixtures thereof Preferred substances include, but are not limited to, hydrogen peroxide and akacid. The structure of akacid has been previously disclosed. See, for example, CAS 374572-91-5(N078)A and Thomas et al., 2005, Chem, Phys. Letters, 402:361-366. Preferably, the bactericidic and/or parasiticidic substance comprises sporicidal activity. The antimicrobial and/or antiparasitic aqueous solution of the present disclosure can include 1-5% (v/v), 1-10% (v/v), or 1-35% (v/v) of the bactericidic and/or parasiticidic substance.

A consideration to take into account when selecting the membrane-opening substance is that the substance preferably is non-toxic (based on the preferred feature that a possible residue on surfaces and objects after the mist treatment should be non-toxic), must be sufficiently water-soluble (so as not to block nozzles and other equipment during the spraying procedure of the mist) and of course that it must provide the wanted opening effect of the cell membrane of bacteria, such as mycobacteria, or spores for providing penetration of the microbicidal and/or parasiticidal substance(s). Because penetration of the cell membrane of mycobacteria can be difficult to achieve, substances effective in the opening of the mycobacteria cell membrane generally will be suitable for achieving a similar effect in spores and most bacteria, fungi, viruses, and parasites. Additionally such a material should not negatively affect the other components of the sprayable aqueous solution. According to the present disclosure, citric acid and/or dimethylsulfone (added to the above mentioned aqueous sprayable hydrogen peroxide solution) are several examples of membrane-opening substance that possess these properties. In an embodiment, it is preferred to use dimethylsulfone as the hydrophilic sulfone.

Examples of organic poly-acids that may be used for membrane opening of mycobacteria and other bacteria, fungi, virus, spores, and parasites according to the present disclosure include, but are not limited to, malic acid, pyruvic acid, tartaric acid, succinic acid, butyric acid, fumaric acid, pyruvic acid, and citric acid. The non-toxic hydrophilic organic poly-acid preferably comprises at least two carboxyl functions and may have a chain length of up to 10 carbon atoms. In an embodiment, the non-toxic hydrophilic organic poly-acid comprises citric acid. To the extent that such poly-acids may exist in different stereoisomeric or tautomeric forms, these are also included in the present invention. Citric acid may exist, for example, as isocitrate and such forms are also included in the present disclosure.

In embodiments, the antimicrobial and/or antiparasitic aqueous solution of the present disclosure comprises 0.1-1% (v/v), 0.1%-4% (v/v), 1-4% (v/v), 0.1%-10% (v/v), or 1-10% (v/v) of the organic polyacid. Since citric acid is highly soluble in water it can be used as such in the aqueous solution. However the preferred concentration of citric acid in the solution that is to be supplied to the relevant surfaces, is 1-10% (v/v), more preferably 1-4% (v/v), this for inter alia avoiding corrosion through acid attack if it should be present in a too strong concentration in the sprayed mist. This is, however, a consideration towards acid-sensitive materials in the surfaces onto which the mist is to settle, and is not meant as a limitation with respect to the concentration that is effective to open the cell membrane of mycobacteria.

Examples of hydrophilic sulfones that may be used for membrane opening of mycobacteria and other bacteria, fungi, virus, spores, and parasites according to the present disclosure include, but are not limited to, di-C₁₋₈-sulfone, di-C₁₋₆-sulfone, di-C_{1— 5}-sulfone, di-C₁₋₄-sulfone, di-C₁₋₃-sulfone, di-C₁₋₂-sulfone, and dimethylsulfone. In embodiments, the antimicrobial and/or antiparasitic aqueous solution of the present disclosure comprises 0.1-1% (v/v), 0.1%-4% (v/v), 1-4% (v/v), 0.1%-10% (v/v), or 1- 10% (v/v) of the hydrophilic sulfone. In an embodiment, the hydrophilic sulfone comprises a non-toxic derivative of di(C₁₋₈)sulfone.

One of the aspects of the present disclosure is to provide a non-toxic composition that includes a di(C₁₋₈)sulfone that is sufficiently soluble in water to provide the membrane-opening effect indicated supra. Dimethylsufone is one example of such a non-toxic material (LD₅₀ (rat, orally)>5 g/kg), this substance may normally be used as a carrier when producing pharmaceutical compositions and agrochemical compositions. The very high water solubility (150 g/l) of dimethylsulfone makes this compound very well suited as an ingredient to aqueous antibacterial or paraciticidal solutions according to the present disclosure.

The exact mechanism of action opening the surface structure of mycobacteria being caused by dimethylsulfone or citric acid, is not known. Extension of the carbon chains in the sulfone compound affects the water solubility of the compound. An extension of the carbon chain beyond C₈ will, however, probably make the compound too poorly soluble in water for it being applicable as an additive in the aqueous solution. The relevant toxicities of carbon chains in the sulfone beyond C₁ will also play a role when selecting the additive, but such considerations may be taken into account by the person skilled in the art in view of the effect of dimethylsulfone. A preferred concentration of dimethylsulfone in the composition according to the present disclosure comprises 1-10% (v/v), more preferably 1-4% (v/v).

Correspondingly, the exact action mechanism of citric acid towards mycobacteria is not known. Corresponding poly-acids may, however, be used as additives separately or to the aqueous solution. Since acids are relevant, the water solubility will not represent any problem, but poly-acids that are used should not be toxic to animals or humans. Poly-acids that naturally enter the metabolism in mammals are a natural choice. Among organic poly-acids (acids including between 2 and 10 COOH-groups) citric acid is preferred.

The antimicrobial and/or antiparasitic aqueous solutions of the present disclosure can is optionally include a polarizing component. The polarizing component depolarizes the cell membrane of bacteria, fungi, viruses, parasites, and spores, making the cell membrane brittle thereby increasing its permeability so that the bactericidic or parasiticidic substance can penetrate into the cell. The polarizing component can also aid in the even distribution of droplet particles of the mist in the environment being treated. The polarizing component may comprise silver ions (Ag⁺, optionally originating from silver nitrate, AgNO₃), gold ions (Au⁺), and/or another non-toxic, non-odorous, and/or non-coloring metal ion. In an embodiment, the polarizing component is an electrically polarizing component, such as Ag⁺ or Au⁺ ions. In some embodiments, the polarizing component comprises a concentration of 10-100 ppm, 10-200 ppm, 10-300 ppm, 10-400 ppm, or 10-500 ppm. In an embodiment, the antimicrobial and/or antiparasitic aqueous solution comprises an Ag⁺ ion concentration of 10-500 ppm. In an embodiment, akacid can function both as a bactericidal substance and a polarizing component.

The antimicrobial and/or antiparasitic aqueous solutions of the present disclosure can also include a water-soluble polymer stabilizer. The stabilizer may be a natural water-soluble polymer, such as rubber arabcum. Other polymers, such as rubber tragacanth, or celluloses, such as carboxy methyl cellulose, may also be used. In an embodiment, the antimicrobial and/or antiparasitic aqueous solution comprises 1 ppm, 1-5 ppm, 1-10 ppm, or 0.5-50 ppm of the stabilizer.

The antimicrobial and/or antiparasitic aqueous solutions of the present disclosure can also include an inorganic acid and/or organic for adjusting the pH of the solutions. Examples of suitable inorganic acids include, but are not limited to, phosphoric acid, hydrochloric acid, nitrous acid, sulfuric acid, and mixtures thereof. Examples of suitable organic acids include, but are not limited to, formic acid, acetic acid, citric acid, oxalic acid, malic acid, tartaric acid, pyruvic acid, and mixtures thereof. In an embodiment, the inorganic acid comprises a concentration of less than 20 ppm, or less than 50 ppm. The pH of the antimicrobial and/or antiparasitic aqueous solutions of the present disclosure generally is from pH 1-7 or from pH 1-5, and optionally can be buffered. In an embodiment, the buffer system comprises a buffer composition comprising said inorganic acids or organic acid and a corresponding salt of said acids for buffering in the pH ranges. The selection of a suitable buffer system may be performed by the person skilled in the art based on known criteria with buffer components that preferably are not be toxic or give any unpleasant smell and/or appearance (in the form of possible residue of e.g. color or other kind of residue) on the surfaces that are treated.

The antimicrobial aqueous solutions of the present disclosure are particularly well suited for killing mycobacterium. Therefore, an important aspect of the present disclosure concerns the addition of a mycobacterium membrane-opening substance to an aqueous solution of a bactericidal or parasiticidal substance that is turned into a dry, electrified mist to be supplied onto surfaces that are to be disinfected. As discussed supra, because penetration of the cell membrane of mycobacteria can be difficult to achieve, substances effective in the opening of the mycobacteria cell membrane generally will be suitable for achieving a similar effect in spores and most bacteria, fungi, viruses, and parasites. Such a mycobacterium membrane-opening substance may be supplied as a separate mist before the aqueous solution is sprayed into the relevant room, may be sprayed as a separate mist at the same time as the aqueous solution is sprayed into the relevant room, or may be added to the aqueous solution directly prior to the spraying being initiated. In an embodiment, the mycobacterium membrane-opening substance is selected among di-C₁₋₈-sulfones, preferably dimethylsulfone, and/or an organic poly-acid, preferably citric acid.

The present disclosure is especially adapted to an aqueous mist process and the spraying of a dry mist. Such a dry mist may be provided, for example, from conventional nozzle devices, e.g. with ultrasound nozzles sold by the company PNR, e.g. the nozzle MAD 0801 B1 or optionally with nozzles such as those disclosed in US patent application 2007/0125882. The selection of suitable nozzles for this purpose may be done by the person skilled in the art based on the desired size intervals of the droplet particles. A fine distribution of the spray is most desirable. The running together of spray particles so that the treated surfaces become moist should be avoided. In embodiments, the diameter of the spherical droplet particles of the mist comprises from about 2 to about 20 microns. In an embodiment, the mean Gauss distribution of the diameter of the spherical droplet particles is from about 7 to about 15 microns. Also the construction of systems for siphoning an antimicrobial and/or antiparasitic aqueous solution of the present disclosure, such as an aqueous hydrogen peroxide solution, from a reservoir for atomization in a nozzle may be performed by a person skilled in the art. An example of a conventional setup is shown in US patent application 2007/0125882, but other setups may also be used.

The dwell time of the mist in the room that is to be disinfected may, to ensure an effective killing of bacteria and microorganisms, lie within the interval from 5 minutes and longer, more preferred from 10 minutes and longer, even more preferred from 15 minutes and longer, most preferred from 60 minutes and longer, and ideally from 120 minutes and longer. The time interval in connection with the treatment starts from the mist being evenly distributed in the available room volume, and the mist needs a time to distribute in the relevant room volume after it has been sprayed from the nozzle. Such time will depend on the number of spraying nozzles, the spray capacity, the size of the room, possible draft or movement of the air masses in the room, etc. and may be measured with a suitable measuring device, e.g. Draeger Polytron 7000 or similar, but will normally lie within an interval so that the exchange of the mist does not surpass 1 room volume per hour. In an embodiment, the mist density is at a minimum 40 ppm or more, and preferably more than 75 ppm.

In such an embodiment, the aqueous mist can include hydrogen peroxide. The hydrogen peroxide concentration in such mists can comprise from 1-5% (v/v), 1-10% (v/v), or 1-35% (v/v), and the bactericidal/mycobactericidal action of this mist corresponds to the oxidation power of the hydrogen peroxide. In other embodiments, the aqueous mist comprises akacid or akacid plus. When using akacid or akacid plus, the concentration of this compound can comprise up to 5% (v/v) or up to 10% (v/v). In an embodiment, the concentration of akacid or akacid plus comprises from 0.1-10% (v/v) or from 1-10% (v/v). In another embodiment, the concentration of akacid or akacid plus comprises from 0.1-5% (v/v) or from 1-5% (v/v). In yet another embodiment, the concentration of akacid or akacid plus comprises from 0.1-0.5% (v/v) or 0.1%-1% (v/v).

When spraying the bactericidal or parasiticidal substance, such as hydrogen peroxide, in the form of a dry mist, the mist particles will remain floating in the room for a long period of time. Even if such a mist disinfecting process is very effective, it is of importance to use the relevant room as soon as possible after the disinfection process has been concluded, i.e. after a sufficient time has passed for the bacteria in the room to have been killed. Since hydrogen peroxide is irritating to the skin and mucous membranes even at low concentrations (1-35% v/v) which is normally used in the present disinfecting mist process, it is preferred to remove the floating hydroxide particles as quickly as possible from the room volume so that the air again may be breathed without any danger or breathing in hydrogen peroxide-containing particles. However, the hydrogen peroxide (which is the only component in the mist that may be harmful) decomposes naturally to water and oxygen until the concentration of hydrogen peroxide eventually is zero. Residues of silver, carboxymethylcellulose and dimethylsulfone are so small that they may be neglected and lie far below the limit for toxicity.

Since hydrogen peroxide is an unstable compound that normally will decompose into oxygen and water, is has conventionally been sufficient to wait for a time long enough for such a decomposition to occur naturally. However, such a passive deactivation of the hydrogen peroxide gives an inconveniently long waiting time before the relevant room may be used again. According to the present disclosure, a solution to this problem has been developed. The solution lies in, subsequent to the treatment time with the hydrogen peroxide mist has ended, sucking the mist back into a neutralization chamber. Since the mist consists of miniscule hydrogen peroxide/water particles, such particles will, when they come in contact with an aqueous medium, dissolve in the medium. Consequently, in its simplest embodiment, it is possible to simply suck the mist through a water container thus dissolving the mist particles in the water. It may also be possible to suck the mist through a demister and/or a filter. Alternatively, it may be possible to lead the hydrogen peroxide particles of the introduced mist through a radiation chamber with (UV/IR) for decomposing the hydrogen peroxide or the aqueous medium may be added a hydrogen peroxide-decomposing enzyme such as catalase for neutralizing the hydrogen peroxide. The other components of the spray mist particles preferably are non-toxic and/or non-irritating and do not need to be neutralized.

In a particular embodiment of the present disclosure, the mist, with particles of a composition according to the present disclosure, is added to a chamber, i.e. a compartment that may seal parts of or the entire body (except the head) of a person inside the compartment. In one embodiment, such a chamber may resemble a sweat box, i.e. a box with a door and including a hole through which the neck of a person may be passed. The hole comprises a suitable sealing material (e.g. rubber, plastic, foam, cloth or other convenient material) for sealing the body or parts of the body of the person inside the sweat box. The sweat box may internally also include a seat such as a stool, chair, bench etc. for the convenience of the person inside the sweat box. The sweat box includes a number of nozzles that are equipped to introduce a mist of an aqueous solution according to the present disclosure (e.g. a composition including hydrogen peroxide at a concentration between about 1 and 35% (v/v) and a di-(C₁₋₈)sulfone and/or organic poly-acid at a concentration between about 1 and 4% (v/v) and optionally an electrically polarizing compound/component and/or a stabilizer and/or akacid plus) inside the sweat box. Such a device is especially meant to treat skin-diseases such as athlete's foot, psoriasis, burn victims (where an absolute sterile environment is required), bacterial sores, fungi, etc. The added antibactericidic composition according to the present disclosure will ensure a completely microorganism-free environment inside the chamber while simultaneously killing all of the germs that may proliferate and fester in the skin of the victim of the relevant disease.

Although hydrogen peroxide is a irritating and highly oxidative substance that should normally not be used directly on exposed tissue (e.g. by brushing, soaking or bathing) the disinfecting process according to the present disclosure uses a dry mist of aqueous solution e.g. containing hydrogen peroxide, i.e. the exposed surfaces remain dry to the touch, and the aqueous particles will not run together either in the air or on the relevant surfaces on account of the polarizing component (the metal ions) of the composition and the miniscule size of the droplets of the mist (see supra). Consequently the patient receiving the mist treatment of the skin with a composition according to the present disclosure, will feel only a slight stinging sensation that is perfectly endurable for the relevant treatment period.

As an example a patient having been treated for a disease or skin condition (sore, abscess, etc.) is to be placed in the “sweat box” and treated with an aqueous dry mist according to the present disclosure within a concentration interval of the mist being 40-100 peak ppm. Such a treatment may also be repeated several times (from one to ten, e.g. two or three) depending on the condition to be treated.

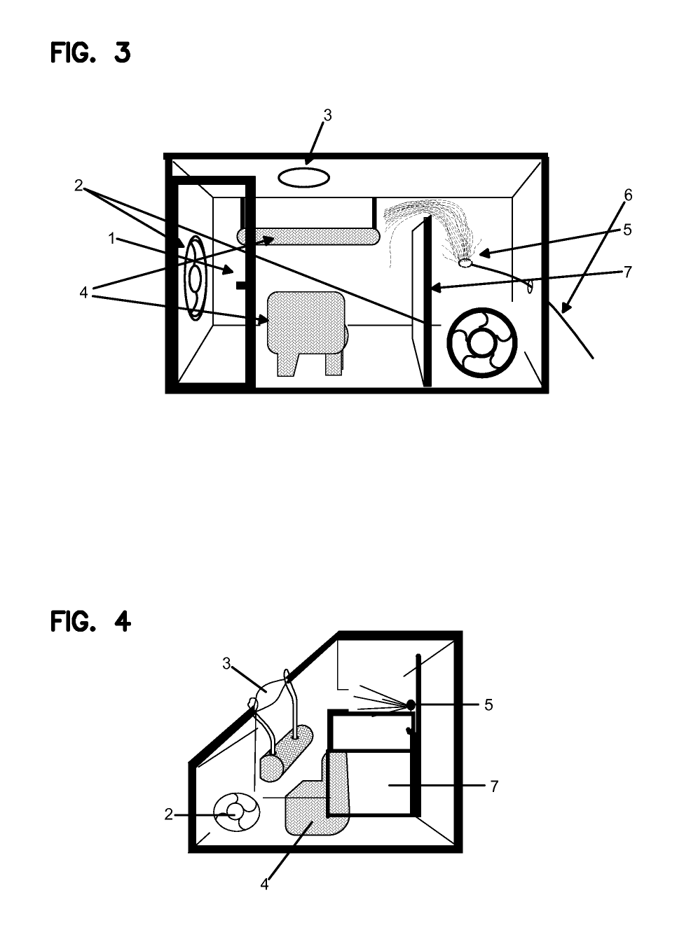

An example of a “sweat box” is depicted in FIGS. 3 and 4 showing an embodiment of such a box observed from the rear and from the side in cross section. The box includes side walls, a floor and a top lid/roof. In one of the side walls there is included a door with air-proofing listings 1. In the door and/or in one of the walls there is located at least one ventilation fan 2. In the lid of the box there is located an aperture 3 for a patient to put his or her head through. The aperture 3 is equipped with a sealing collar. Inside the box there is also located a chair or other seat 4 and optionally also an arm-support for the patient's comfort. The box is also equipped with a nozzle 5 for introducing a mist of the composition according to the present disclosure into the box. The nozzle 5 is connected to a supply hose 6 for the composition according to the present disclosure. Preferably the box is equipped internally with a dividing wall 7 to avoid spraying the composition directly onto the skin of the patient. The dividing wall 7 is located between the chair 4 and the nozzle 5. The box may also be equipped with devices for monitoring the mist concentration inside the box and duration of the treatment.

The composition according to the present disclosure may also be used for disinfecting vehicles such as ambulances or other emergency vehicles such as ambulance boats, ambulance helicopters, etc.

In an ambulance, boat, helicopter etc. it is important to reduce the risk of infection since such vehicles are used in constantly different states of emergencies where contamination and transfer of diseases is very possible. However, if a mist-disinfection procedure according to the present disclosure is to be performed it is important to ensure a completely or substantially air-tight sealing of the vehicle compartment(s). For this purpose it has been developed a separate sealing system to be placed around the joints and abutting surfaces/edges around vehicle doors and optionally windows for ensuring a sufficiently gas-impenetrable sealing for the time the mist-disinfection according to the present disclosure is being performed.

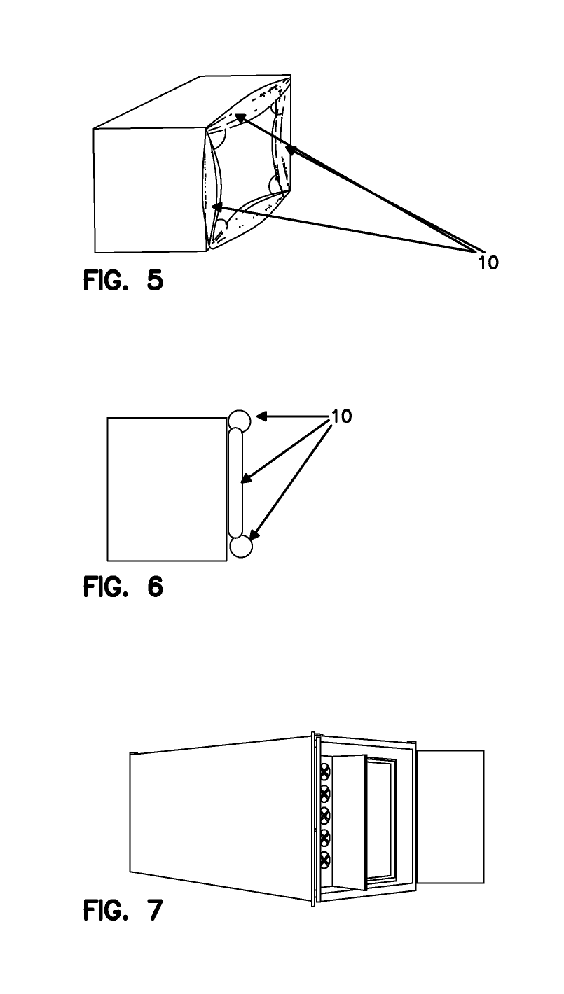

An embodiment of such a sealing device as indicated supra is depicted in FIGS. 5 and 6. The sealing device forms a sluice/tunnel to be secured to the relevant vehicle opening, such as shown in FIG. 7. The sealing device comprises a ballooning frame 10 that fits inside the opening of the relevant vehicle and that will expand when pressurized. The ballooning frame is made of a soft, pliable material such as plastic or rubber that will not be or is insignificantly affected by the hydrogen peroxide mist that is sprayed into the vehicle through the sluice. On account of the ballooning effect of the frame, the sluice may be fitted to different types of vehicle openings. The ballooning frame may be connected to a tunnel including a supply nozzle for the dry spray according to the present disclosure, and such a tunnel may also include measuring devices for monitoring the dry mist concentration and the duration of the dry mist treatment. The edge of the tunnel (the sluice) is made of an air-tight pocket/cell. Here it is welded an inlet for gas or air to be connected to pressurized gas/air. When the gas/air enters the sluice the device will expand and abut against the frame of the opening of the relevant vehicle so it becomes air-tight, but opening into the tunnel. The tunnel is then filled with the composition according to the present disclosure as a dry mist expanding into the compartment of the vehicle that is to be decontaminated, e.g. the patient compartment of an ambulance.

### EXAMPLES

The examples provided infra are given to illustrate the present disclosure but without limiting it in any way.

### Example 1

The spraying of a conventional dry hydrogen peroxide mist, not containing any dimethylsulfone or citric acid, was performed in a non-sterile room in Ullevaal University Hospital to investigate the effect of such a mist on Mycobacterium tuberculosis. The room was sealed by closing the doors and windows, and sources for draft/air-suction were removed. The spraying of a dry 5% H₂O₂ mist containing silver-ions (concentration 50 ppm) and arab rubber (concentration 1% v/v) was performed by spraying thrice with an even spacing between the sprayings during 2 hours per period (totally 6 hours) where the top concentration of the mist at the first spraying was 45 ppm, at the second spraying was 55 ppm and at the third spraying 60 ppm. After treatment a bacterial smear (20 in number) from samples taken from 6 different locations in the treated room was done. Growth of Mycobacterium was detected in all samples (20/20). Control samples taken from the same locations in the room prior to the mist treatment showed growth in most of the samples (19/20).

A corresponding test taken in another room (with the same parameters as indicated supra) showed growth of Mycobacterium in 19/20 of the control samples and 16/20 of the samples subsequent to the hydrogen peroxide mist treatment.

### Example 2

This example was conducted corresponding to example 1, but with the difference that the number of treatments with hydrogen peroxide mist were increased to six, where the top concentration of the hydrogen peroxide mist particles at the fourth to sixth spray treatment was 65, 70 and 75 ppm, respectively. The results displayed growth in the control samples in 13/20 and in 10/20 of the exposed samples after 4 weeks. Corresponding numbers taken from another room displayed growth in 17/20 of the control samples and 10/20 of the exposed samples after 3 weeks.

The results from test 1 and 2 show that a mist treatment with hydrogen peroxide according to the prior art is not sufficient to obtain disinfection of the relevant rooms with respect to Mycobacterium.

### Example 3

In this example, an aqueous hydrogen peroxide solution with a concentration of 5% was used. The other components were as in example 1, except that dimethylsuofone was io added at a concentration of 3% (v/v) to the spray composition. The mist treatment was performed through three cycles with a top level of hydrogen peroxide mist particles of 106.7 ppm per top over a period of 240 minutes with each mist treatment distributed evenly over this time interval. During the test the temperature was 23° C. and a relative humidity of 28.8%. The spore-killing properties against bacterial spores of this composition was proven by there not being found bacterial growth in any of the treated samples (no mycobacteria either), while in the 3 controls there was growth shown in all of the control samples.

Also a second parallel test was conducted in the same manner, but in a different room with a diffusion of 23 cubics, verified these results, where there was not detected any bacterial growth in any of the samples taken from the treated room, while bacterial growth occurred in all of the control samples (3 in number).

### Example 4

A dry hydrogen peroxide mist of the present disclosure was tested to determine the ability of the mist to kill bacteria associated with hospital acquired infections and decontaminate materials commonly found in the hospital environment. The dry mist contained 6% hydrogen peroxide, 70 ppm silver ions, and 0.5% citric acid. Stainless steel, paper, and cloth discs were inoculated in triplicate with 20 μl suspension (10⁸ CFU/ml) of Staphylococcus aureus, Enterococcus faecium, Salmonella enterica infantis, Candida albicans, and Pseudomonoas aeruginosa, and Geobacillus stearothermophilus, respectively. G. stearothermophilus spores are commonly used as a challenge organism for sterilization and checking of sterilization cycles. G. stearothermophilus is thermophilic and its spores are known to be among the toughest to destroy in a sterilization procedure. Treatments capable of killing G. stearothermophilus spores are therefore generally considered to be suitable for killing most spores, bacteria, viruses, and parasites. For this reason, G. stearothermophilus was included in the testing of the dry hydrogen peroxide mist.

The inoculated discs were moved to a test room that was subsequently treated by spraying the interior of the room with the dry hydrogen peroxide mist. The test room was subjected to three cycles of the mist treatment. A first control set of samples was processed immediately after start of the treatment. A second control set of samples was left in the test room and processed after the treatment.

After the treatment, all of the sample discs were removed from the test room and the inoculated organisms were released from the sample discs. Each stainless steel and cloth disc was put into a tube with 2 ml 0.9% NaCl solution and vortexed for 1 min. The paper discs were treated with ultrasound. After washing, the samples were diluted in series and spotted in duplicate on a plate. The plates were incubated overnight at 35° C. and colonies were enumerated the following day. The G. stearothermophilus plates were incubated at 56° C. C. albican colonies were counted after 48 hr incubation.

The results of the experiment are shown in Table 1 below. CFU/ml recovered from inoculated control discs at the start of treatment (t=0), from inoculated control discs after treatment (t=3), and from experimental discs (t=3 exposed) are shown in the table. The detection limit is 25 CFU/ml. Zero values in the table therefore represent values below 25 CFU/ml. The number of S. aureus CFU from the paper discs after treatment may be less than the detection limit as the plate count contained an aberrant result.

TABLE 1

| | | t = 0 | t = 3 | t = 3 |
|---|---|---|---|---|
| Organism | Material | unexposed | unexposed | exposed |
| Staphylococcus | steel | 4.1E3 | 8.8E2 | 8.3E0 |
| aureus | paper | 2.0E4 | 3.1E3 | 8.3E1(0) |
| | cloth | 7.1E3 | 3.6E3 | 0 |
| Enterococcus | steel | 3.0E5 | 3.4E5 | 0 |
| faecium (VRE) | paper | 5.8E5 | 4.4E5 | 0 |
| | cloth | 1.9E5 | 1.1E5 | 0 |
| Salmonella | steel | 6.5E3 | 5.3E3 | 0 |
| enterica Infantis | paper | 5.7E3 | 5.3E2 | 0 |
| | cloth | 2.4E4 | 2.8E4 | 0 |
| Candida albicans | steel | 3.1E4 | 9.1E3 | 0 |
| | paper | 8.9E4 | 2.5E4 | 0 |
| | cloth | 5.6E4 | 3.8E4 | 0 |
| Pseudomonas | steel | 4.2E3 | 8.5E2 | 0 |
| aeruginosa | paper | 8.0E2 | 5.0E1 | 0 |
| | cloth | 5.3E3 | 4.3E2 | 0 |
| Geobacillus stea- | steel | nd | 4.0E5 | 5.8E1 |
| rothermophilus | paper | nd | 1.6E6 | 0 |
| | cloth | nd | 7.1E5 | 2.5E1 |

As shown in Table 1, the dry hydrogen peroxide mist according to the present disclosure is capable of killing microorganisms associated with hospital acquired infections on materials commonly found in the hospital environment and provides for the decontamination of surfaces in the area treated with the mist. The significant killing of G. stearothermophilus spores, which are commonly used as a challenge organism for sterilization and checking of sterilization cycles, further demonstrates that the treatment is suitable for killing most spores, bacteria, viruses, and parasites for surface decontamination.

We claim:

1. An antimicrobial aqueous composition for use as an electrified dry spray mist, said composition comprising 1-35% (v/v) hydrogen peroxide, 1-10% (v/v) organic poly-acid, a stabilizer, and optionally at least one polarizing component.

2. The composition according to claim 1, wherein said composition further comprises 0.1-0.5 (v/v) akacid.

3. The composition according to claim 1, wherein said organic poly-acid is an acid comprising 2 to 10 COOH-groups.

4. The composition according to claim 1, wherein said composition comprises 1-4% (v/v) organic poly-acid.

5. The composition according to claim 1, wherein said polarizing component comprises metallic ions.

6. The composition according to claim 5, wherein said metallic ions comprise Ag⁺-ions.

7. The composition according to claim 1, wherein said stabilizer comprises carboxymethylcellulose.

8. A method for disinfecting a surface of an object, comprising spraying the composition according to claim 1 as an electrified aqueous mist onto an external surface of said object or into an air space surrounding said object or defined by the surface of said object such that the composition contacts the surface of the object.

9. The method according to claim 8, wherein said composition kills bacteria or bacterial spores on the surface of said object.

10. The method according to claim 9, wherein the bacteria comprises mycobacteria.

11. A method for disinfecting a room or vehicle, comprising spraying the composition according to claim 1 as an electrified mist onto surfaces in the room or vehicle or into an air space defined by interior surfaces of the room or vehicle such that the composition contacts the interior surfaces of the room or vehicle.

12. The method according to claim 11, wherein said composition kills bacteria or bacterial spores in the room or vehicle.

13. The method according to claim 12, wherein the bacteria comprises mycobacteria.

14. The method according to claim 11, wherein the room is a hospital room, operating theatre, emergency room, sterile room, or containment room.

15. The method according to claim 11, wherein the vehicle is an emergency vehicle.

16. The composition according to claim 3, wherein said organic poly-acid comprises citric acid, malic acid, succinic acid, fumaric acid, or pyruvic acid.

17. The composition according to claim 1, wherein said organic poly-acid is citric acid.

18. The composition according to claim 4, wherein said organic poly-acid is citric acid.

19. The composition according to claim 5, wherein said metallic ions comprise Au⁺-ions.

20. The method according to claim 15, wherein the emergency vehicle is an ambulance.
